(19) United States
(12) Patent Application Publication
(10) Pub. No.: US 2023/0210669 A1
(43) Pub. Date: Jul. 6, 2023

(54) ARTIFICIAL ARTICULATION HAVING STABLE FIXING MEANS FOR UNICOMPARTMENTAL KNEE ARTHROPLASTY

(71) Applicants: Kyoung Tak KANG, Seoul (KR); TJC life Co., Ltd., Seoul (KR)

(72) Inventors: Jin Ah LEE, Seoul (KR); Yong Gon KOH, Seoul (KR); Kyoung Tak KANG, Seoul (KR)

(21) Appl. No.: 17/567,616

(22) Filed: Jan. 3, 2022

(51) Int. Cl. A61F 2/38; A61F 2/30

(52) U.S. Cl. CPC A61F 2/3868; A61F 2/30771; A61F 2/30907; A61F 2002/30878; A61F 2002/30891; A61F 2002/30317; A61F 2002/30326; A61F 2002/30784; A61F 2002/3093

(57) ABSTRACT

An artificial articulation having stable fixing means for unicompartmental knee arthroplasty is stably attached to the top of a tibia in order to permit a sliding motion of an artificial joint attached to the bottom of a femur. The artificial articulation includes an implant hat part which a curved surface structure that makes surface contact with the artificial joint attached to the bottom of the femur is formed on an upper surface thereof, and protrusions protruding by a predetermined height in a direction of the top of the tibia are formed in large numbers on a lower surface thereof, and a distally extending stem configured to be formed to protrude downward by a predetermined length from the lower surface of the implant hat part, and to be inserted into the tibia by a predetermined depth from the top of the tibia and attached thereto.

### BACKGROUND OF THE INVENTION

### 1. Field of the Invention

The present disclosure relates to an artificial articulation used in unicompartmental knee arthroplasty (UKA) and, more particularly, to an artificial articulation attached to the top of the tibia (shinbone) in order to permit a sliding motion of an artificial joint attached to the bottom of the femur (thighbone).

### 2. Description of Related Art

When the knee joint is highly deformed due to diseases such as gonarthrosis or chronic rheumatoid arthritis, surgery is performed to replace the knee joint with artificial components to restore normal function.

Knee replacement surgery, also known as knee arthroplasty, is a surgical procedure performed when the knee joint is damaged by various diseases or traumatic injuries, etc., resulting in impairment of the knee joint so severe that it is no longer possible to carry out normal daily activities. Also, the surgery is performed on patients who cannot be treated with drug therapy or physical therapy or by other surgical methods.

Knee arthroplasty according to the conventional art is an operation that inserts an artificial joint made of safe ceramic and plastic materials into the body of a patient.

Knee arthroplasty is an operation that reduces pain in the knee joint and allows almost normal movement of the knee joint, while at the same time restoring stability of the knee joint and correcting deformation to enable normal living.

Thanks to continuous development, an artificial joint according to the conventional art is similar to a normal knee joint by using various materials so that it will not be damaged or worn out even after long-term use.

In the past knee arthroplasty was aimed at eliminating pain, but now, artificial joints that can be used for a long time and can be bent (i.e., flexed) well have been developed and used in surgery to not only relieve pain but also maintain joint function.

Among artificial joints are ceramic-type artificial joints that extend the lifespan of artificial joints, high-flexion artificial joints that can be bent a lot, and female-type artificial joints tailored to women's knee j oints.

There are two main types of knee replacement surgeries: total knee arthroplasty (TKA) where the entire joint is replaced with artificial surfaces; and unicompartmental knee arthroplasty (UKA), also called partial knee replacement, where only a portion of the knee damaged by knee arthritis is replaced.

The artificial joint used in UKA consists of a femoral component attached to the bottom of the femur, and a tibial plate attached to the top of the tibia to permit a sliding motion of the femoral component.

Here, a screw-shaped protrusion structure is used as shown in FIG. 1 in order for the tibial plate to be stably mounted on top of the tibia.

However, in such a case, a structure corresponding to the screw-shaped protrusion should be separately formed on top of the tibia.

Also, in the case of the tibial plate according to the conventional art, there is no configuration that can effectively induce osseointegration, so the protruding length and shape of the screw-shaped protrusion are modified for stable fixation, but there is a problem in that the operation takes a lot of time due to the reason that a structure corresponding to the screw-shaped protrusion should be formed on top of the tibia.

Thus, there is a need for a technique capable of solving the problems of the conventional art.

Documents of Related Art
(Patent Document 1) U.S. Pat. No. 4,298,992 (Nov. 10, 1981)

### SUMMARY OF THE INVENTION

Accordingly, the present disclosure has been made keeping in mind the above problems occurring in the related art, and the present disclosure is intended to provide an artificial articulation configured to be stably attached to the top of the tibia in order to permit a sliding motion of an artificial joint attached to the bottom of the femur.

In order to achieve the above objective, according to one aspect of the present disclosure, there is provided an artificial articulation that is attached to the top of the tibia in order to permit a sliding motion of an artificial joint attached to the bottom of the femur. The artificial articulation may include: an implant hat part which a curved surface structure that makes surface contact with the artificial joint attached to the bottom of the femur is formed on an upper surface thereof, and protrusions protruding by a predetermined height in a direction of the top of the tibia are formed in large numbers on a lower surface thereof; and a distally extending stem configured to be formed to protrude downward by a predetermined length from the lower surface of the implant hat part, and to be inserted into the tibia by a predetermined depth from the top of the tibia and attached thereto.

In an embodiment of the present disclosure, the protrusions formed on the lower surface of the implant hat part may have pointed needle-like structures so as to be inserted into the tibia by a predetermined depth through the top surface of the tibia.

In an embodiment of the present disclosure, the protrusions formed on the lower surface of the implant hat part may be arranged in large numbers in a predetermined pattern over an area corresponding to the top surface of the tibia.

In an embodiment of the present disclosure, the protrusions formed on the lower surface of the implant hat part may be arranged in the large numbers over the area corresponding to the top surface of the tibia, and an arrangement density of the protrusions may be set differently depending on a magnitude and a location of load applied from the artificial joint attached to the bottom of the femur.

In an embodiment of the present disclosure, the protrusions formed on the lower surface of the implant hat part may be arranged in large numbers over the area corresponding to the top surface of the tibia, and a protruding height of the protrusions may be set differently depending on a magnitude and a location of load applied from the artificial joint attached to the bottom of the femur.

In an embodiment of the present disclosure, at least one osseointegration hole for inducing osseointegration, after the artificial articulation is mounted on a patient subjected to surgery, may be formed on the lower side of the implant hat part.

In an embodiment of the present disclosure, the osseointegration holes may be arranged in large numbers in a predetermined pattern along an outer peripheral surface of the lower side of the implant hat part.

In an embodiment of the present disclosure, the osseointegration holes may be formed in large numbers along the outer peripheral surface of the lower side of the implant hat part, and an arrangement density of the protrusions may be set differently depending on a magnitude and a location of load applied from the artificial joint attached to the bottom of the femur.

In an embodiment of the present disclosure, the lower surface of the implant hat part may have a mesh structure in which a plurality of pores are formed.

In this case, the inside of the implant hat part may be a hollow structure.

In an embodiment of the present disclosure, the side surface and the lower surface of the implant hat part 110 of the artificial articulation may have porous structures in which a plurality of pores are formed, and the outer surface of the distally extending stem may have a porous structure in which a plurality of pores are formed.

In an embodiment of the present disclosure, at least one osseointegration hole for inducing osseointegration, after the artificial articulation is mounted on a patient subjected to surgery, may be formed on the side surface of the distally extending stem.

As described above, the artificial articulation according to the present disclosure can be stably attached to the top of the tibia by having the implant hat part with a specific structure and the distally extending stem.

Also, the artificial articulation according to the present disclosure can be stably attached to the top of the tibia by arranging the protrusions with a specific structure on the lower surface of the implant hat part in a predetermined pattern, by varying the arrangement density, or by varying the protruding height.

In addition, the artificial articulation according to the present disclosure makes it possible to control the degree of osseointegration in accordance with designer's intention by arranging the osseointegration holes in a predetermined pattern on the lower side of the implant hat part or by varying the arrangement density, so that the artificial articulation can be stably attached to the top of the tibia.

Also, the artificial articulation according to the present disclosure may induce effective osseointegration by having the lower surface of the implant hat part in a mesh structure or by having the inside of the implant hat part in a hollow structure, so that the artificial articulation can be stably attached to the top of the tibia.

In addition, the artificial articulation according to the present disclosure may induce effective osseointegration by having the side surface and the lower surface of the implant hat part in porous structures in which a plurality of pores are formed, and by having an outer surface of the distally extending stem in a porous structure in which a plurality of pores are formed, so that the artificial articulation can be stably attached to the top of the tibia.

Further, the artificial articulation according to the present disclosure may induce effective osseointegration by having at least one osseointegration hole for inducing osseointegration, after the artificial articulation is mounted on a patient subjected to surgery, on a side surface of the distally extending stem, so that the artificial articulation can be stably attached to the top of the tibia.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a photograph showing an artificial joint according to the conventional art;

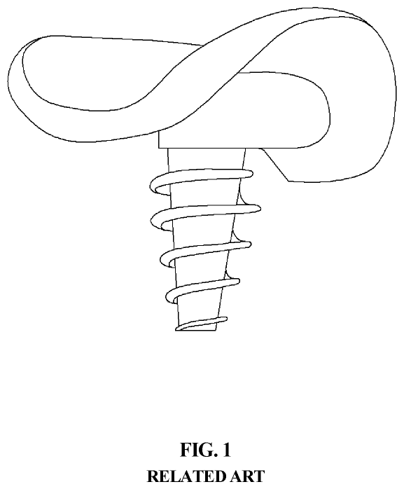

FIG. 2 is a front view showing an artificial articulation according to an embodiment of the present disclosure;

FIGS. 3A and 3B are schematic views showing the arrangement of protrusions formed on the lower surface of an implant hat part according to embodiments of the present disclosure;

FIG. 4 is a perspective view showing an artificial articulation according to another embodiment of the present disclosure;

FIG. 5 is a vertical cross-sectional view showing the artificial articulation shown in FIG. 4;

FIG. 6 is a perspective view showing an artificial articulation according to yet another embodiment of the present disclosure; and

FIG. 7 is a vertical cross-sectional view showing the artificial articulation according to yet to another embodiment of the present disclosure.

### DETAILED DESCRIPTION OF THE INVENTION

Hereinafter, preferred embodiments of the present disclosure will be described in detail with reference to the accompanying drawings. All terms and words used herein should not be construed as being limited to conventional or dictionary meanings but should be interpreted as meanings and concepts consistent with the technical spirit of the present disclosure.

Throughout the specification, when a member is referred to be located “on” another member, it should be understood that the member may be in contact with another member, but yet another member may exist in between. Throughout the specification, when a part “comprises” or “includes” a component, it means that other components may be further comprised or included, rather than excluding other components, unless otherwise stated.

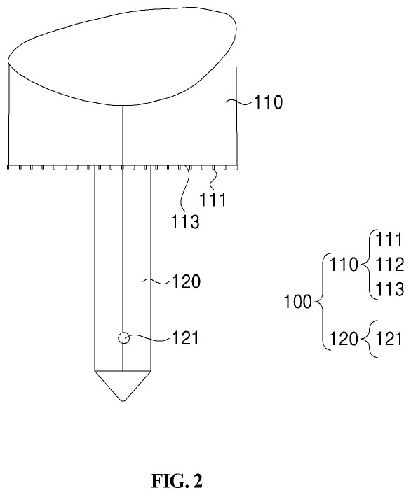

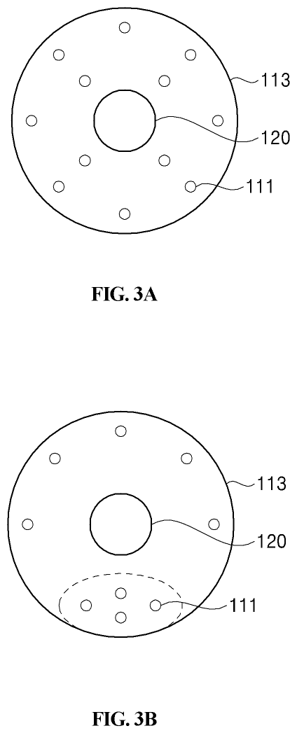

FIG. 2 is a front view showing an artificial articulation according to an embodiment of the present disclosure, and FIGS. 3A and 3B are schematic views showing the arrangement of protrusions formed on the lower surface of an implant hat part according to embodiments of the present disclosure.

Referring to these drawings, the artificial articulation according to the embodiment is attached to the top of a tibia in order to permit a sliding motion of an artificial joint attached to the bottom of a femur, and may be configured to include an implant hat part 110 with a specific structure and a distally extending stem 120.

To be specific, on the upper surface of the implant hat part 110, a curved surface structure that makes surface contact with the artificial joint attached to the bottom of the femur may be formed, while on the lower surface of the implant hat part 110, protrusions 111 protruding by a predetermined height in the direction of the top of the tibia may be formed in large numbers.

A distally extending stem 120 is configured to be formed to protrude downward by a predetermined length from the lower surface of the implant hat part 110, and to be inserted into the tibia by a predetermined depth from the top of the tibia and attached thereto.

The protrusions 111 formed on the lower surface of the implant hat part 110 may have a structure that is stably bound to the top surface of the tibia, and preferably, they may have pointed needle-like structures so as to be inserted into the tibia by a predetermined depth through the top surface of the tibia.

As shown in FIG. 3A, the protrusions 111 formed on the lower surface of the implant hat part 110 may be arranged in large numbers in a predetermined pattern over an area corresponding to the top surface of the tibia.

Also, the protrusions 111 formed on the lower surface of the implant hat part 110 are arranged in large numbers over the area corresponding to the top surface of the tibia, and an arrangement density of the protrusions 111 may be set differently depending on a magnitude and a location of load applied from the artificial joint attached to the bottom of the femur. (see the dotted line area shown in FIG. 3B)

In some cases, the protrusions 111 formed on the lower surface of the implant hat part 110 are arranged in large numbers over the area corresponding to the top surface of the tibia, and a protruding height of the protrusions 111 may be set differently depending on a magnitude and a location of load applied from the artificial joint attached to the bottom of the femur. In this case, although not shown in FIGS. 3A and 3B, it is preferable to set the protruding height of the protrusions 111 relatively longer at locations where the magnitude of the load is greater than that of the surrounding area.

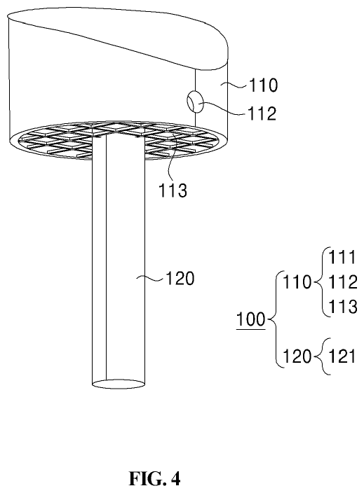

FIG. 4 is a perspective view showing an artificial articulation according to another embodiment of the present disclosure.

Referring to FIG. 4, at least one osseointegration hole 112 for inducing osseointegration, after the artificial articulation is mounted on a patient subjected to surgery, may be formed on the lower side of the implant hat part 110 according to the embodiment.

In some cases, the osseointegration holes 112 may be arranged in large numbers in a predetermined pattern along an outer peripheral surface of the lower side of the implant hat part 110.

Also, the osseointegration holes 112 may be arranged in large numbers along an outer peripheral surface of the lower side of the implant hat part 110, and an arrangement density of the osseointegration holes 112 may be set differently depending on a magnitude and a location of load applied from the artificial joint attached to the bottom of the femur.

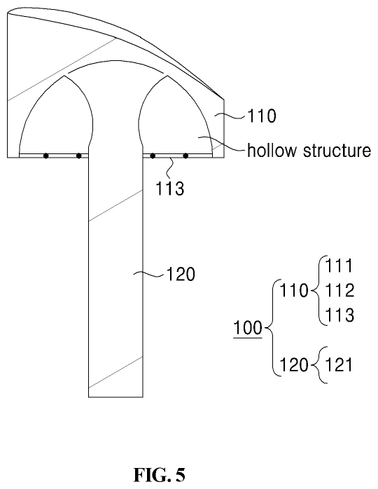

Meanwhile, as shown in FIG. 4, the lower surface 113 of the implant hat part 110 may have a mesh structure in which a plurality of pores are formed so as to effectively induce stable fixation of the artificial articulation 100 by inducing osseointegration after the artificial articulation 100 is mounted. Here, as shown in FIG. 5, it is preferable that the inside of the implant hat part 110 be a hollow structure.

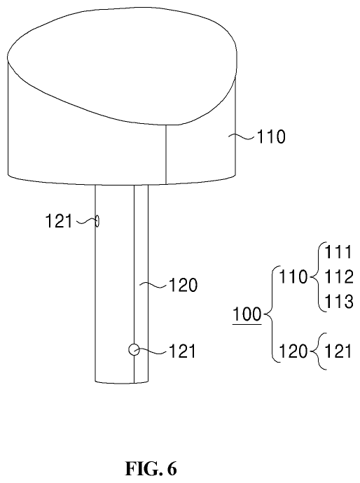

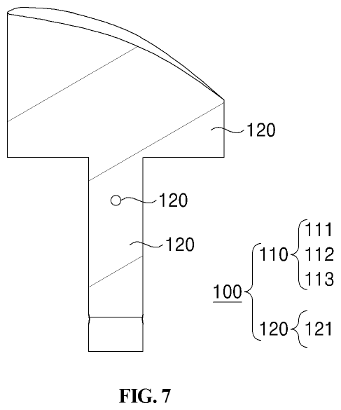

FIG. 6 is a perspective view showing an artificial articulation according to yet another embodiment of the present disclosure, and FIG. 7 is a vertical cross-sectional view showing the artificial articulation according to yet another embodiment of the present disclosure.

Referring to these drawings, the side surface and the lower surface 113 of the implant hat part 110 of the artificial articulation 100 according to the embodiment may have porous structures in which a plurality of pores are formed. In the same way, an outer surface of the distally extending stem 120 may have a porous structure in which a plurality of pores are formed.

As such, osseointegration can be effectively induced, and as a result, the artificial articulation 100 can be stably attached to the top of the tibia.

In addition, at least one osseointegration hole 112 for inducing osseointegration is formed on the side surface of the distally extending stem 120 to effectively induce osseointegration.

As described above, the artificial articulation according to the present disclosure can be stably attached to the top of the tibia by having the implant hat part 110 with a specific structure and the distally extending stem 120.

Also, the artificial articulation according to the present disclosure can be stably attached to the top of the tibia by arranging the protrusions 111 with a specific structure on the lower surface of the implant hat part 110 in a predetermined pattern, by varying the arrangement density, or by varying the protruding height.

In addition, the artificial articulation according to the present disclosure makes it possible to control the degree of osseointegration in accordance with designer's intention by arranging the osseointegration holes 112 in a predetermined pattern on the lower side of the implant hat part 110 or by varying the arrangement density, so that the artificial articulation can be stably attached to the top of the tibia.

Also, the artificial articulation according to the present disclosure may induce effective osseointegration by having the lower surface 113 of the implant hat part 110 in a mesh structure or by having the inside of the implant hat part 110 in a hollow structure, so that the artificial articulation can be stably attached to the top of the tibia.

In addition, the artificial articulation according to the present disclosure may induce effective osseointegration by having a side surface and the lower surface 113 of the implant hat part 110 in porous structures in which a plurality of pores are formed, and by having an outer surface of the distally extending stem 120 in a porous structure in which a plurality of pores are formed, so that the artificial articulation can be stably attached to the top of the tibia.

Further, the artificial articulation according to the present disclosure may induce effective osseointegration by having at least one osseointegration hole 112 for inducing osseointegration, after the artificial articulation is mounted on a patient subjected to surgery, on a side surface of the distally extending stem 120, so that the artificial articulation can be stably attached to the top of the tibia.

In the above detailed description of the present disclosure, only specific embodiments thereof have been described. Hence, it should be understood that the present disclosure is not to be limited to the particular form mentioned in the detailed description, but rather to cover all modifications, equivalents, and substitutions falling within the spirit and scope of the present disclosure as defined by the appended claims.

That is, the present disclosure is not limited to the specific embodiments and descriptions described above, and any person skilled in the art to which the present disclosure pertains may implement various modifications without departing from the gist of the present disclosure as claimed in the claims, and such modifications shall fall within the scope of protection of the present disclosure.

What is claimed is:

1. An artificial articulation (100) attached to a top of a tibia in order to permit a sliding motion of an artificial joint attached to a bottom of a femur, the artificial articulation (100) comprising:
an implant hat part (110) in which a curved surface structure that makes surface contact with the artificial joint attached to the bottom of the femur is formed on an upper surface thereof, and protrusions (111) protruding by a predetermined height in a direction of the top of the tibia are formed in large numbers on a lower surface thereof, and
a distally extending stem (120) configured to protrude downward by a predetermined length from the lower surface of the implant hat part (110), and to be inserted into the tibia by a predetermined depth from the top of the tibia and attached thereto,
wherein the protrusions (111) formed on the lower surface of the implant hat part (110) are arranged in the large numbers over an area corresponding to a top surface of the tibia, and
an arrangement density of the protrusions (111) varies depending on a magnitude and a location of load applied from the artificial joint attached to the bottom of the femur.

2. The artificial articulation of claim 1, wherein the protrusions (111) formed on the lower surface of the implant hat part (110) have pointed needle-like structures so as to be inserted into the tibia by a predetermined depth through the top surface of the tibia.

3. The artificial articulation of claim 1, wherein the protrusions (111) formed on the lower surface of the implant hat part (110) are arranged in the large numbers in a predetermined pattern over the area corresponding to the top surface of the tibia.

4. An artificial articulation (100) attached to a top of a tibia in order to permit a sliding motion of an artificial joint attached to a bottom of a femur, the artificial articulation (100) comprising:
an implant hat part (110) in which a curved surface structure that makes surface contact with the artificial joint attached to the bottom of the femur is formed on an upper surface thereof, and protrusions (111) protruding by a predetermined height in a direction of the top of the tibia are formed in large numbers on a lower surface thereof, and
a distally extending stem (120) configured to protrude downward by a predetermined length from the lower surface of the implant hat part (110), and to be inserted into the tibia by a predetermined depth from the top of the tibia and attached thereto,
wherein the protrusions (111) formed on the lower surface of the implant hat part (110) are arranged in the large numbers over an area corresponding to a top surface of the tibia, and
a protruding height of the protrusions (111) varies depending on a magnitude and a location of load applied from the artificial joint attached to the bottom of the femur.

5. The artificial articulation of claim 1, wherein at least one osseointegration hole (112) for inducing osseointegration, after the artificial articulation is mounted on a patient subjected to surgery, is formed on a lower side of the implant hat part (110).

6. The artificial articulation of claim 5, wherein the osseointegration holes (112) are arranged in large numbers in a predetermined pattern along an outer peripheral surface of the lower side of the implant hat part (110).

7. The artificial articulation of claim 5, wherein the osseointegration holes (112) are formed in large numbers along an outer peripheral surface of the lower side of the implant hat part (110), and an arrangement density of the osseointegration holes (112) varies depending on a magnitude and a location of load applied from the artificial joint attached to the bottom of the femur.

8. The artificial articulation of claim 1, wherein the lower surface of the implant hat part (110) has a mesh structure in which a plurality of pores are formed.

9. The artificial articulation of claim 8, wherein an inside of the implant hat part (110) has a hollow structure.

10. The artificial articulation of claim 1, wherein a side surface and the lower surface of the implant hat part (110) have porous structures in which a plurality of pores are formed, and an outer surface of the distally extending stem (120) has a porous structure in which a plurality of pores are formed.

11. The artificial articulation of claim 1, wherein at least one osseointegration hole (112) for inducing osseointegration, after the artificial articulation is mounted on a patient subjected to surgery, is formed on a side surface of the distally extending stem (120).
